(19) United States
(12) Patent Application Publication
(10) Pub. No.: US 2024/0415977 A1
(43) Pub. Date: Dec. 19, 2024

(54) OPHTHALMIC DRUG DELIVERY CARRIER AND METHOD FOR PREPARING THE SAME

(71) Applicant: Chang Gung University, Taoyuan City (TW)

(72) Inventors: Jui-Yang Lai, Taoyuan City (TW); Chia-Jung Yang, Taoyuan City (TW)

(21) Appl. No.: 18/499,169

(22) Filed: Oct. 31, 2023

(30) Foreign Application Priority Data
Jun. 14, 2023 (TW) 112122246

(51) Int. Cl. A61K 47/69; A61K 9/00; A61K 31/05; A61K 47/64; A61P 27/02

(52) U.S. Cl. CPC A61K 47/6925; A61K 9/0048; A61K 31/05; A61K 47/645; A61P 27/02

(57) ABSTRACT

An ophthalmic drug delivery carrier includes a resveratrol-encapsulating poly(ε-caprolactone) (PCL) nanoparticle, metformin grafted on a surface of the resveratrol-encapsulating PCL nanoparticle, and a transacting activator of transcription (TAT) peptide grafted on the surface of the resveratrol-encapsulating PCL nanoparticle. A method for preparing the ophthalmic drug delivery carrier and a method for alleviating macular degeneration using the ophthalmic drug delivery carrier are also provided.

### CROSS-REFERENCE TO RELATED APPLICATION

This application claims priority to Taiwanese Invention patent application Ser. No. 11/212,2246, filed on Jun. 14, 2023, and incorporated by reference herein in its entirety.

### SEQUENCE LISTING XML

The Sequence Listing submitted concurrently herewith with a file name of “CGUN-016-101 SEQUENCE LISTING.xml,” a creation date of Oct. 31, 2023, and a size of 3,162 bytes, is part of the specification and is incorporated by reference in its entirety.

### FIELD

The disclosure relates to an ophthalmic drug delivery carrier and a method for preparing the same. The disclosure also relates to a method for alleviating macular degeneration using the ophthalmic drug delivery carrier.

### BACKGROUND

Macular degeneration refers to an ophthalmological disease which is caused by damage to the macula in the center of the retina, and may result in blurred vision or no vision in the center of the visual field. Since aging plays a crucial role in the development of such ophthalmological disease, macular degeneration is also known as age-related macular degeneration (AMD) or senile macular degeneration, and can be additionally classified into two forms based on clinical symptoms, that is, dry form and wet form of macular degeneration.

The dry form of macular degeneration (i.e., atrophic macular degeneration) is the more common form of the two, where drusen are usually found to be deposited under the retina and geographic atrophy of the retinal pigment epithelium (RPE) may be discovered as well. The dry form of macular degeneration, when continues to get worse, may turn into the wet form of macular degeneration.

The wet form of macular degeneration (i.e., neovascular macular degeneration (NMD) or exudative macular degeneration) is associated with an abnormal growth of new blood vessels. When such new blood vessels grow into the retina, they tend to induce retinal hemorrhage, leading to swelling of the retina and even vision loss, in which sudden blindness or acute blindness accounts for 90%.

Methods used clinically for treating macular degeneration may include laser photocoagulation, transpupillary thermotherapy (TTT), photodynamic therapy (PDT), anti-angiogenesis therapy, and nutritional supplement therapy. Among the above-mentioned methods, the most commonly used one is anti-angiogenesis therapy, which inhibits angiogenesis via intravitreal injection of anti-vascular endothelial growth factor (anti-VEGF) into the vitreous body, but the requirement of frequent injections often results in serious adverse side effects (such as subconjunctival hemorrhage, high intraocular pressures, intraocular infections, and retinal detachment) in a subject.

In spite of the aforesaid, there is still a need for those skilled in the art to develop a method that can more efficiently deliver an ophthalmic drug to the retina tissue and that has greater efficacy in treatment of macular degeneration.

### SUMMARY

Therefore, in a first aspect, the present disclosure provides an ophthalmic drug delivery carrier that can alleviate at least one of the drawbacks of the prior art. The ophthalmic drug delivery carrier includes:
a resveratrol-encapsulating poly(ε-caprolactone) (PCL) nanoparticle;
metformin grafted on a surface of the resveratrol-encapsulating PCL nanoparticle; and
a transacting activator of transcription (TAT) peptide grafted on the surface of the resveratrol-encapsulating PCL nanoparticle.

In a second aspect, the present disclosure provides a method for preparing the aforesaid ophthalmic drug delivery carrier that can alleviate at least one of the drawbacks of the prior art. The method includes:
mixing an emulsion containing resveratrol with PCL and a surfactant for double emulsification so as to form a mixture, followed by subjecting the mixture to centrifugation so as to obtain a precipitate;
subjecting the precipitate to an amination reaction with a diamine solution, so as to obtain a resveratrol-encapsulating PCL nanoparticle;
subjecting a TAT peptide to a carboxylation reaction so as to obtain a TAT peptide having a carboxyl group;
subjecting metformin to a carboxylation reaction so as to obtain metformin having a carboxyl group; and
subjecting the resveratrol-encapsulating PCL nanoparticle to a grafting reaction with the TAT peptide having the carboxyl group and the metformin having the carboxyl group, thereby obtaining the ophthalmic drug delivery carrier.

In the grafting reaction, the resveratrol-encapsulating PCL nanoparticle, the TAT peptide having the carboxyl group, and the metformin having the carboxyl group are present in a weight ratio ranging from 1:1.5:0.15 to 1:1.5:1.5.

In a third aspect, the present disclosure provides a method for alleviating macular degeneration that can alleviate at least one of the drawbacks of the prior art and that includes administrating to a subject in need thereof a pharmaceutical composition containing the aforesaid ophthalmic drug delivery carrier.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the Fourier Transform Infrared Spectroscopy (FTIR) spectra of ophthalmic drug delivery carriers 1 to 3, i.e., T/M-R-PCL-NP 1, T/M-R-PCL-NP 2, and T/M-R-PCL-NP 3 of Example 1, infra.

FIG. 2 shows the grafting rates of TAT peptide and metformin determined in the T/M-R-PCL-NP 1, the T/M-R-PCL-NP 2, and the T/M-R-PCL-NP 3 of Example 1, infra.

FIG. 3 shows the percentage of cell migration determined in each group of Example 3, infra, in which the symbol “*” represents p<0.05 (compared with the normal control group) and the symbol “#” represents p<0.05 (compared with the comparative group).

FIG. 4 shows the relative fluorescence intensity of retinal tissues determined in each group of Example 4, infra, in which the symbol “*” represents p<0.05 (compared with the normal control group) and the symbol “#” represents p<0.05 (compared with the experimental group).

FIG. 5 shows the amplitudes of the a-wave and the b-wave determined in each of the normal control group, the pathological control group, the comparative groups 1 and 2, and the experimental group of Example 5, infra, in which the symbol “*” represents p<0.05 (compared with the normal control group), the symbol “#” represents p<0.05 (compared with the pathological control group), and the symbol “†” represents p<0.05 (compared with the comparative groups 1 and 2).

FIG. 6 shows the vascular front density of retinal tissues determined in each group of Example 5, infra, in which the symbol “*” represents p<0.05 (compared with the normal control group), the symbol “#” represents p<0.05 (compared with the pathological control group), and the symbol “†” represents p<0.05 (compared with the comparative groups 1 to 3).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

While conducting experiments, the applicant has found that a resveratrol (RSV)-encapsulating poly(ε-caprolactone) (PCL) nanoparticle having a transacting activator of transcription (TAT) peptide grafted on a surface thereof exhibits excellent retinal penetration property, so that the resveratrol encapsulated thereby and metformin (Met) grafted on the surface thereof, both of which are immiscible to each other as conventionally known to those skilled in the art, can be simultaneously delivered to the retinal tissue, thereby synergistically exerting the effect of alleviating age-related macular degeneration (AMD).

Therefore, the present disclosure provides an ophthalmic drug delivery carrier, which includes:
a resveratrol (RSV)-encapsulating poly(ε-caprolactone) (PCL) nanoparticle;
metformin grafted on a surface of the resveratrol-encapsulating PCL nanoparticle; and
a transacting activator of transcription (TAT) peptide grafted on the surface of the resveratrol-encapsulating PCL nanoparticle.

In certain embodiments, the RSV-encapsulating PCL nanoparticle may have a particle size ranging from 80 nm to 150 nm. In an exemplary embodiment, the RSV-encapsulating PCL nanoparticle has a particle size ranging from 100 nm to 120 nm.

In certain embodiments, a grafting ratio of the TAT peptide to the metformin may range from 10:90 to 90:10. In certain embodiments, the grafting ratio of the TAT peptide to the metformin may be selected from 80:20, 40:60, and 15:85.

In certain embodiments, a grafting rate of the TAT peptide may range from 10% to 90%. In certain embodiments, the grafting rate of the TAT peptide may range from 15% to 80%.

In certain embodiments, a grafting rate of the metformin may range from 10% to 90%. In certain embodiments, the grafting rate of the metformin may range from 20% to 85%.

The present disclosure also provides a method for preparing the aforesaid ophthalmic drug delivery carrier, which includes:
mixing an emulsion containing resveratrol with PCL and a surfactant for double emulsification so as to form a mixture, followed by subjecting the mixture to centrifugation so as to obtain a precipitate;
subjecting the precipitate to an amination reaction with a diamine solution, so as to obtain a resveratrol-encapsulating PCL nanoparticle;
subjecting a TAT peptide to a carboxylation reaction so as to obtain a TAT peptide having a carboxyl group;
subjecting metformin to a carboxylation reaction so as to obtain metformin having a carboxyl group; and
subjecting the resveratrol-encapsulating PCL nanoparticle to a grafting reaction with the TAT peptide having the carboxyl group and the metformin having the carboxyl group, thereby obtaining the ophthalmic drug delivery carrier.

In the grafting reaction, the resveratrol-encapsulating PCL nanoparticle, the TAT peptide having the carboxyl group and the metformin having the carboxyl group are present in a weight ratio ranging from 1:1.5:0.15 to 1:1.5:1.5.

In an exemplary embodiment, the resveratrol-encapsulating PCL nanoparticle, the TAT peptide having the carboxyl group, and the metformin having the carboxyl group are present in a weight ratio of 1:1.5:0.75.

According to the present disclosure, the resveratrol and the metformin may be commercially available products, or may be prepared by chemical synthesis using techniques well known to those skilled in the art.

Alternatively, the resveratrol may be obtained from a plant source using an isolation and purification method well known to those skilled in the art.

According to the present disclosure, the TAT peptide may be a commercially available product, or may be obtained using at least one of the following methods: chemical synthesis, in vitro transcription, and in vivo expression.

According to the present disclosure, the TAT peptide contains an amino acid sequence of RKKRRQRRR (SEQ ID NO: 1).

According to the present disclosure, the double emulsification may be conducted using techniques well known to those skilled in the art. In this regard, those skilled in the art may refer to, e.g., Marchal-Heussler L. et al. (1993), Pharm. Res., 10:386-390.

It can be comprehended that the conditions for conducting the double emulsification may vary with some factors such as the amount of the emulsion containing the resveratrol, the PCL and the surfactant in order to achieve the most desired emulsification result. Selection of the conditions for conducting the double emulsification is within the expertise and routine skills of those skilled in the art.

According to the present disclosure, the emulsion containing the resveratrol may be obtained by mixing the resveratrol with a reagent selected from the group consisting of TiO₅, heptane, dimethyl sulfoxide (DMSO), ethanol, chloroform, sodium dodecyl sulfate (SDS), xanthan gum, a polysorbate, and combinations thereof. In an exemplary embodiment, the reagent is TiO₅.

According to the present disclosure, the surfactant may be selected from the group consisting of poloxamer F68 (Pluronic® F68), poloxamer F127 (Pluronic® F127), Tween® 60, polyoxyl 35 castor oil, and combinations thereof. In an exemplary embodiment, the surfactant is poloxamer F68 (Pluronic® F68).

According to the present disclosure, the amination reaction may be conducted using techniques well known to those skilled in the art. In this regard, those skilled in the art may refer to, e.g., Zhu Y. et al. (2002), Biomacromolecules, 3:1312-1319.

It can be understood that the conditions for conducting the amination reaction may vary with some factors, such as the type and the amount of an amine group donor (i.e., the diamine solution) and an amine group acceptor in order to achieve the most desired amination effect. Selection of the conditions for conducting the amination reaction is within the expertise and routine skills of those skilled in the art.

According to the present disclosure, the amination reaction may be carried out at a temperature ranging from 35° C. to 37° C. for a time period ranging from 1 hour to 24 hours. In an exemplary embodiment, the amination reaction is carried out at 37° C. for 1 hour.

According to the present disclosure, the diamine solution may be selected from the group consisting of a 1,6-hexanediamine solution, an ethylenediamine solution, a propylenediamine solution, a phenyl diamidophosphate solution, and combinations thereof. In an exemplary embodiment, the diamine solution is a 1,6-hexanediamine solution.

According to the present disclosure, the carboxylation reaction (of each of the TAT peptide and the metformin) may be conducted using techniques well known to those skilled in the art. In this regard, those skilled in the art may refer to, e.g., Koniev O. and Wagner A. et al. (2015), Chem. Soc. Rev., 44:5495-5551.

It can be understood that the conditions for conducting the carboxylation reaction may vary with some factors such as the amount of a carboxyl group donor (e.g., maleimide-PEG-COOH and NHS-PQ-COOH) and a carboxyl group acceptor (i.e., the TAT peptide or the metformin) in order to achieve the most desired carboxylation effect. Selection of the conditions for conducting the carboxylation reaction is within the expertise and routine skills of those skilled in the art.

According to the present disclosure, the carboxylation reaction may be carried out at a temperature ranging from 23° C. to 27° C. for a time period ranging from 1 hour to 24 hours. In an exemplary embodiment, the carboxylation reaction of the TAT peptide is carried out at 25° C. for 24 hours, and the carboxylation reaction of the metformin is carried out at 25° C. for 2 hours.

According to the present disclosure, the NHS-PQ-COOH contains an amino acid sequence of GGGPQGIWGQGK (SEQ ID NO: 2).

According to the present disclosure, the grafting reaction may be conducted using techniques well known to those skilled in the art. In this regard, those skilled in the art may refer to, e.g., Ostadhossein F. et al. (2018), Bioconjug. Chem., 29:3913-3922.

It can be comprehended that conditions for conducting the grafting reaction may vary with some factors such as the amount of the resveratrol-encapsulating PCL nanoparticle, the TAT peptide having the carboxyl group, and the metformin having the carboxyl group in order to achieve the most desired grafting effect. Selection of the conditions for conducting the grafting reaction is within the expertise and routine skills of those skilled in the art.

According to the present disclosure, the grafting reaction may be carried out at a temperature ranging from 16° C. to 26° C. for a time period ranging from 18 hours to 24 hours. In an exemplary embodiment, the grafting reaction is carried out at 25° C. for 24 hours.

Due to the fact that the ophthalmic drug delivery carrier has been proved, through animal experiments, to be effective in relieving impaired retinal function and abnormal proliferation of blood vessels occurring in rats which are caused by age-related macular degeneration (AMD), the ophthalmic drug delivery carrier is hence expected to be useful as a therapeutic agent for alleviating macular degeneration.

Therefore, the present disclosure provides a method for alleviating macular degeneration, which includes administrating to a subject in need thereof a pharmaceutical composition containing the aforesaid ophthalmic drug delivery carrier.

As used herein, the term “macular degeneration” refers to age-related macular degeneration (AMD) (also known as senile macular degeneration) or juvenile macular degeneration (JMD). In addition, the AMD may be dry AMD (also known as atrophic AMD), wet AMD (also known as neovascular AMD), and exudative AMD.

As used herein, the term “alleviating” or “alleviation” refers to at least partially preventing, reducing, treating, ameliorating, relieving or controlling one or more clinical signs of a disease or disorder, and lowering, stopping or reversing the progression of severity regarding the condition or symptom being treated.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the pharmaceutical composition may be formulated into a dosage form suitable for intraocular administration or topical ophthalmic administration using technology well known to those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents (e.g., a sterile water), buffers (e.g., an ophthalmic balanced salt solution, phosphate buffered saline (PBS), Ringer's solution and Hank's solution), emulsifiers, suspending agents, decomposers, pH adjusting agents, stabilizing agents, chelating agents, preservatives, diluents, absorption delaying agents, liposomes, lubricants, and the like. The choice and amount of the aforesaid agents are within the expertise and routine skills of those skilled in the art.

According to the present disclosure, the dosage form suitable for topical ophthalmic administration includes, but is not limited to, drops, emulsions, gels, ointments, creams, sprays, micelles, and suspensions.

According to the present disclosure, the dosage form suitable for intraocular administration includes, but is not limited to, an injection, e.g., a sterile aqueous solution, a dispersion or an emulsion.

The pharmaceutical composition according to the present disclosure may be administered via one of the following routes: subtenon injection, intravitreal injection, intracameral injection, intra-retinal injection, subretinal injection, and suprachoroidal injection.

The dose and frequency of administration of the pharmaceutical composition of the present disclosure may vary depending on the following factors: the severity of the illness or disorder to be treated, routes of administration, and age, physical condition and response of the subject to be treated. In general, the pharmaceutical composition may be administered in a single dose or in several doses.

The present disclosure will be further described by way of the following examples. However, it should be understood that the following examples are intended solely for the purpose of illustration and should not be construed as limiting the present disclosure in practice.

### EXAMPLES

### General Experimental Materials

1. The materials and sources thereof used in the following experiments are shown in Table 1 below.

TABLE 1

| Material | | Source |
|---|---|---|
| Cell- | Protamine | Sigma-Aldrich |
| penetrating | Poly(arginine) | |
| peptide | Poly(lysine) | |
| (CPP) | Poly(serine) | |
| | Transacting activator of | |
| | transcription (TAT) peptide | |

| | |
|---|---|
| Poly(ε-caprolactone) (PCL) | |
| (a number average molecular weight (Mn) of | |
| approximately 10 kDa) | |
| Resveratrol (RSV) | |
| Metformin (Met) | |
| 1,6-hexanediamine | |
| Maleimide-PEG-COOH | |
| 1-ethyl-3-(3-dimethylaminopropyl)carbodiimide | |
| hydrochloride (EDC) | |
| Bevacizumab (Avastin ®) | |
| TiO₅ | Henkel |
| NHS-PQ-COOH | Biotools Co., Ltd. |

TAT peptide: RKKRRQRRR (SEQ ID NO: 1)
PQ-COOH: GGGPQGIWGQGK (SEQ ID NO: 2)

### 2. Experimental Animals:

Sprague Dawley (SD) rats (10 to 20 weeks old, with a body weight of approximately 250 g to 400 g) used in the following experiments were purchased from National Laboratory Animal Center, R.O.C. All the experimental rats were housed in an animal room with an independent air conditioning system under the following laboratory conditions: an alternating 12-hour light and 12-hour dark cycle, a temperature maintained at a range of 20° C. to 24° C., and a relative humidity maintained at a range of 55% to 65%. Furthermore, water and food were provide ad libitum for all the experimental rats. All experimental procedures involving the experimental rats were in compliance with the guidelines of the Institutional Animal Care and Use Committee (IACUC) of Chang Gung University and the Association for Research in Vision and Ophthalmology (ARVO).

### General Experimental Procedures

### 1. Statistical Analysis:

The experimental data of all the test groups are expressed as mean ±standard deviation (SD), and were analyzed using one-way analysis of variance (one-way ANOVA), so as to evaluate the differences between the groups. Statistical significance is indicated by p<0.05.

### Example 1. Preparation of Ophthalmic Drug Delivery Carrier According to the Disclosure

A. Synthesis of Resveratrol-Encapsulating poly(ε-Caprolactone) (PCL) Nanoparticles

Resveratrol-encapsulating PCL nanoparticles were synthesized using a double emulsification method according to the following procedures.

First, 40 mg of the resveratrol and 0.5 mL of the TiO₅ were evenly mixed by stirring so as to form an emulsion. Subsequently, 100 mg of the PCL (prepared in 50 ml of acetone) was added to the emulsion, and then 250 mg of poloxamer F68 (Pluronic® F68) (serving as a surfactant; prepared in 50 ml of deionized water) was added therein, followed by conducting an ultrasonication treatment with a power of 200 W for 30 seconds, so as to obtain a dispersion. Next, the dispersion was concentrated to a volume of 5 mL under vacuum, followed by centrifugation at 65400 g for 10 minutes, so as to obtain a supernatant and a precipitate. After that, the supernatant was removed and the precipitate was washed with deionized water two times. Next, 10 wt % of a 1,6-hexanediamine solution (serving as a diamine solution; prepared in 2-propanol) was added to the precipitate so as to allow an amination reaction to proceed at 37° C. for 1 hour, followed by washing with deionized water five times to remove unreacted 1,6-hexanediamine, thereby obtaining a mixture. Afterward, the mixture was dried in a vacuum oven at 30° C. for 24 hours, thereby obtaining the resveratrol-encapsulating PCL nanoparticles (hereinafter abbreviated as “R-PCL-NP”).

### B. Synthesis of a TAT Peptide Having a Carboxyl Group

First, 10 μM of the TAT peptide (prepared in 0.25 ml of an acetonitrile/dimethylformamide mixture) and 10 μM of the maleimide-PEG-POOH (prepared in 1 mL of chloroform) were mixed so as to allow a carboxylation reaction to proceed at 25° C. in the dark overnight, thereby obtaining a carboxylated product. Next, the carboxylated product was subjected to precipitation using 3 mL of an ice-cold mixture of diethyl ether and methanol (volume ratio of the diethyl ether to the methanol is 80:20), followed by centrifugation at 1800 g for 10 minutes so as to obtain a supernatant and a precipitate, and then the supernatant was removed.

Subsequently, the precipitate was washed with chloroform three times, followed by drying in a vacuum oven at 30° C. for 24 hours, thereby obtaining a TAT peptide having a carboxyl group (hereinafter abbreviated as “TAT-PEG-COOH”).

### C. Synthesis of Metformin Having a Carboxyl Group

First, 20 μM of the NHS-PQ-COOH (prepared in 50 μL of dimethyl sulfoxide (DMSO)) and 20 μM of the metformin (prepared in 200 μL of deionized water) were mixed so as to allow a carboxylation reaction to proceed at 25° C. in the dark for 2 hours, thereby obtaining a carboxylated product. After that, the carboxylated product was added to a mixture containing 1000 μL of ethanol and 3 M of sodium acetate (volume ratio of the ethanol to the sodium acetate is 9:1), followed by centrifugation at 3600 g and 4° C. for 20 minutes so as to obtain a supernatant and a precipitate, and then the supernatant was removed. Next, the precipitate was washed with 70% cold ethanol two times, followed by drying in a vacuum oven at 30° C. for 24 hours, thereby obtaining metformin having a carboxyl group (hereinafter abbreviated as “Met-PQ-COOH”).

### D. Synthesis of Ophthalmic Drug Delivery Carrier

Each of the ophthalmic drug delivery carriers 1 to 3, abbreviated as T/M-R-PCL-NP 1, T/M-R-PCL-NP 2, and T/M-R-PCL-NP 3, respectively, was prepared using the corresponding grafting solution shown in Table 2 and according to the procedures described below.

First, 1 g of the R-PCL-NP prepared in section A of this example was dispersed in 5 mL of a 2-(N-morpholino) ethanesulfonic acid (MES) buffer containing 0.26 g of the EDC, followed by stirring for 6 hours so as to obtain a first mixture. Afterward, the first mixture was mixed with 5 ml of the grafting solution containing the TAT-PEG-COOH prepared in section B of this example and the Met-PQ-COOH prepared in section C of this example (the TAT-PEG-COOH and the Met-PQ-COOH were prepared in 5 mL of a MES buffer), thereby obtaining a second mixture. Subsequently, the second mixture was left standing at room temperature for 24 hours so as to allow a grafting reaction to proceed, and then was left standing again at 50° C. for 1 hour to allow precipitation to occur, thereby forming a first supernatant and a first precipitate. Next, the first supernatant (containing residues of the TAT-PEG-COOH and the Met-PQ-COOH that were unreacted) was collected for serving as a test solution used in Section C of Example 2. The first precipitate was washed with deionized water three times, followed by centrifugation at 5400 g for 10 minutes, thereby forming a second supernatant and a second precipitate. After that, the second supernatant was removed, and the second precipitate thus obtained was resveratrol-encapsulating PCL nanoparticles grafted with the TAT-PEG-COOH and the Met-PQ-COOH (i.e., the ophthalmic drug delivery carrier). Finally, a respective one of the ophthalmic drug delivery carrier 1 to 3 (i.e., T/M-R-PCL-NP 1, T/M-R-PCL-NP 2, and T/M-R-PCL-NP 3, respectively) was subjected to lyophilization at-50° C. for 24 hours so as to form a lyophilized powder of the T/M-R-PCL-NP 1, a lyophilized powder of the T/M-R-PCL-NP 2, and a lyophilized powder of the T/M-R-PCL-NP 3.

TABLE 2

| Ophthalmic drug | Grafting | Amount (g) | |
|---|---|---|---|
| delivery carrier | solution | TAT-PEG-COOH | Met-PQ-COOH |
| T/M-R-PCL-NP 1 | 1 | 1.5 | 0.15 |
| T/M-R-PCL-NP 2 | 2 | 1.5 | 0.75 |
| T/M-R-PCL-NP 3 | 3 | 1.5 | 1.5 |

### Example 2. Property Evaluation of the Ophthalmic Drug Delivery Carrier According to the Disclosure

### A. Determination of Particle Size

A respective one of the lyophilized powder of the T/M-R-PCL-NP 1, the lyophilized powder of the T/M-R-PCL-NP 2, and the lyophilized powder of the T/M-R-PCL-NP 3, in appropriate amount thereof, was subjected to dynamic light scattering (DLS) using a Zetasizer Nano ZS analyzer (Malvern Instruments, Worcestershire, UK), followed by measurement of particle size thereof.

The results show that each of the T/M-R-PCL-NP 1, T/M-R-PCL-NP 2, and T/M-R-PCL-NP 3 has a particle size ranging from 80 nm to 150 nm.

### B. Fourier Transform Infrared Spectroscopy (FTIR) Analysis

A respective one of the lyophilized powder of the T/M-R-PCL-NP 1, the lyophilized powder of the T/M-R-PCL-NP 2, and the lyophilized powder of the T/M-R-PCL-NP 3, in appropriate amount thereof, was subjected to FTIR analysis using an attenuated total reflection (ATR)-FTIR spectrometer (FT-730) (Horiba, Japan), and FTIR spectra were collected over a wavenumber ranging from 3500 cm⁻¹ to 1000 cm⁻¹ at a resolution of 8 cm⁻¹.

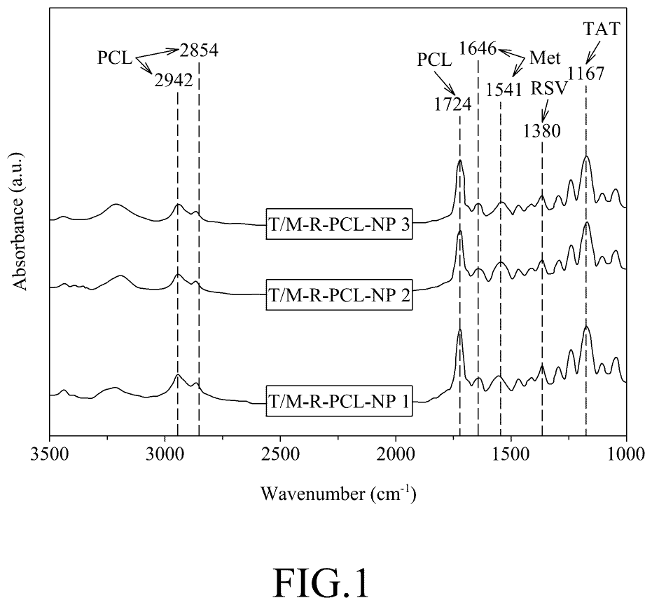

Referring to FIG. 1, the respective one of the T/M-R-PCL-NP 1, T/M-R-PCL-NP 2, and T/M-R-PCL-NP 3 had characteristic peaks of the PCL at wavenumbers of 2942 cm⁻¹, 2854 cm⁻¹, and 1724 cm⁻¹, characteristic peaks of the metformin at wavenumbers of 1646 cm⁻¹ and 1541 cm⁻¹, a characteristic peak of the resveratrol at wavenumber of 1380 cm⁻¹, and a characteristic peak of the TAT peptide at wavenumber of 1167 cm⁻¹, indicating the all the T/M-R-PCL-NP 1, T/M-R-PCL-NP 2, and T/M-R-PCL-NP 3 indeed had the resveratrol encapsulated therein and the metformin and the TAT peptide grafted thereon.

### C. Determination of Grafting Rate

Each of the three test solutions obtained in Section D of Example1 was subjected to weight measurement, and was then mixed with an appropriate amount of a ninhydrin solution, followed by heating for 20 minutes, so that the residue of the TAT-PEG-COOH that was unreacted in the test solution could react with the ninhydrin solution to produce color, thereby forming a mixture. After that, the mixture was cooled down to room temperature, and was then diluted with 95% ethanol, followed by measurement of absorbance value at a wavelength of 570 nm (OD₅₇₀) using an UV-visible spectrophotometer (Thermo Scientific). The absorbance value thus measured was converted into a concentration (ng/ml) according to a standard curve of standard products with different glycine concentrations that was known in advance and absorbance values thereof correspondingly, so as to determine a content of the TAT peptide grafted on the R-PCL-NP.

In addition, each of the lyophilized powder of the T/M-R-PCL-NP 1, the lyophilized powder of the T/M-R-PCL-NP 2, and the lyophilized powder of the T/M-R-PCL-NP 3 was subjected to measurement of absorbance value at a wavelength of 233 nm (OD₂₃₃) using the UV-visible spectrophotometer. The absorbance value thus measured was converted into a concentration (ng/ml) according to a standard curve of standard products with different metformin concentrations that was known in advance and absorbance values thereof correspondingly, so as to determine a content of the metformin grafted on the R-PCL-NP.

Subsequently, the grating rates (%) of the TAT peptide and the metformin were calculated using the following Equations (1) and (2):

$$\begin{matrix}A = [ C / ( C + D ) ] \times 100 & ( 1 ) \\ B = [ D / ( C + D ) ] \times 100 & ( 2 )\end{matrix}$$

where A=grafting rate of TAT peptide (%)
B=grafting rate of metformin (%)
C=content of TAT peptide measured on respective R-PCL-NP
D=content of metformin measured on respective R-PCL-NP

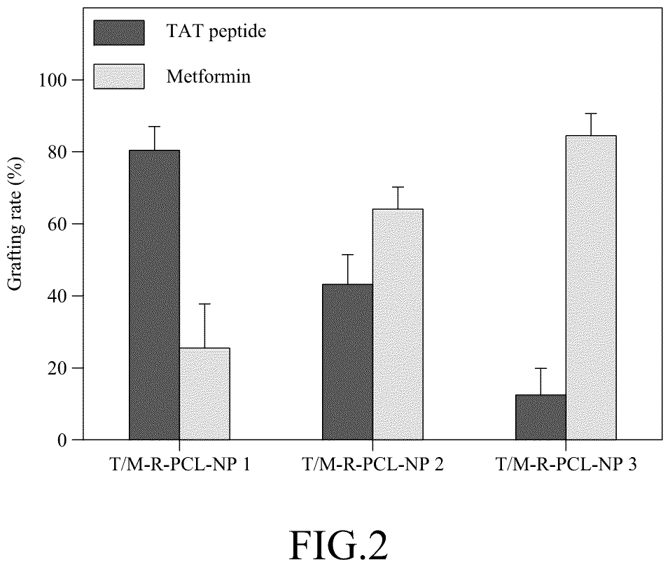

Referring to FIG. 2, the grafting rate of the metformin, from the highest to the lowest are: T/M-R-PCL-NP 3, T/M-R-PCL-NP 2, and T/M-R-PCL-NP 1. Moreover, the grafting rate of the TAT peptide, from the highest to the lowest are: T/M-R-PCL-NP 1, T/M-R-PCL-NP 2, and T/M-R-PCL-NP 3. These results indicate that the grafting rate of the metformin would gradually increase with the increase in the amount of the metformin used; however, the grafting rate of the TAT peptide would gradually decrease with the increase in the amount of the metformin used. In addition, it can be further inferred from FIG. 2 that the grating ratios of the TAT peptide to the metformin on the T/M-R-PCL-NP 1, T/M-R-PCL-NP 2 and T/M-R-PCL-NP 3 were 80:20, 40:60, and 15:85, respectively.

### Example 3. Evaluation of Antiangiogenic Effect of the Ophthalmic Drug Delivery Carrier According to the Disclosure

In this example, the applicant utilized a cell migration assay to evaluate the antiangiogenic effect of the ophthalmic drug delivery carrier according to the disclosure. Moreover, for comparison purpose, the R-PCL-NP obtained in Example 1 was also subjected to the experiments of this example.

### Experimental Materials

### 1. Source and Culture of Human Umbilical Vein Endothelial Cells (HUVECs):

The HUVECs used in this example were purchased from Lifeline Cell Technology. The HUVECs were cultivated in VasucLife® EnGS endothelial cell growth (ECG) medium (Lifeline Cell Technology, LLC) an incubator with culture conditions set at 37° C. and 5% CO₂. Thereafter, medium replacement was carried out approximately every 2-3 days. Cell passage was performed when the cultured cells reached approximately 80% of confluence.

### Experimental Procedures

First, the HUVECs were divided into five groups, including a normal control group, a comparative group, and three experimental groups (experimental groups 1 to 3). The HUVECs in each group were seeded in a respective well of an 8-well culture plate containing the VasucLife® EnGS ECG medium, and were then cultivated in an incubator with culture conditions set at 37° C., 5% CO₂. When the cultured cells reached approximately 80% of confluence, a pipette tip was used to scrape the cultured cells along a diameter of each well, so as to create a gap free of cell attachment. Subsequently, the culture medium was removed, and the cell culture was washed with a serum-free medium twice, followed by filling the wells of the experimental groups 1 to 3 with VasucLife® EnGS ECG mediums containing an appropriate amount of T/M-R-PCL-NP 1, T/M-R-PCL-NP 2 and T/M-R-PCL-NP 3, respectively, filling the well of the comparative group with a VasucLife® EnGS ECG medium containing an appropriate amount of R-PCL-NP, and filling the well of the normal control group with the VasucLife® EnGS ECG medium (i.e., without any R-PCL-NP therein).

After that, the cell culture in each group was cultivated in an incubator (37° C. and 5% CO₂) for 24 hours, and then the number of the cultured cells which migrated into the gap was observed and counted using a phase contrast microscope (Nikon) at a magnification of 4×. Next, the percentage of cell migration (%) in each group was calculated using the following Equations (3):

$$E = ( F / G ) \times 100 \qquad ( 3 )$$

where E=percentage of cell migration (%)
F=number of the cultured cells migrating into the gap in each group
G=number of the cultured cells migrating into the gap in the normal control group

The data thus obtained were analyzed according to the procedures as described in Section 1 of General Experimental Procedures.

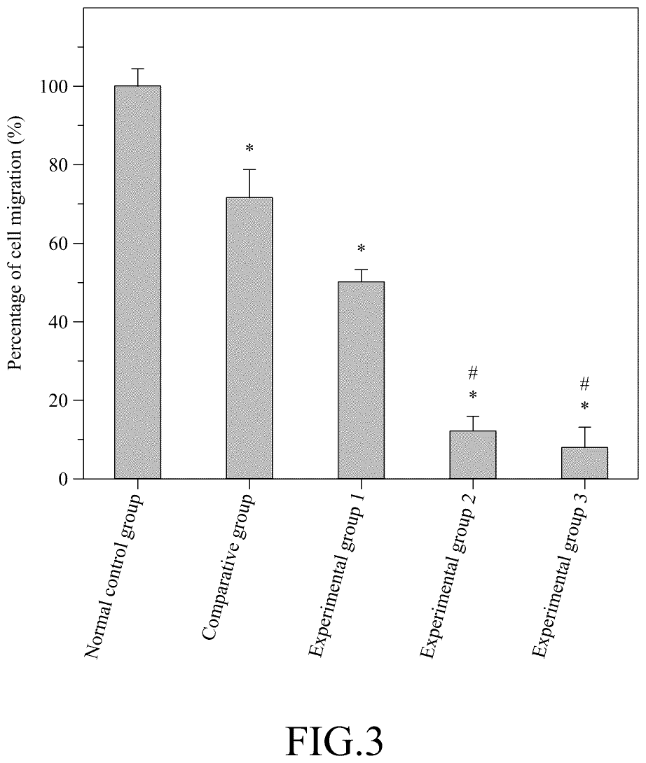

Referring to FIG. 3, compared with the percentage of cell migration of the normal control group, the percentage of cell migration in each of the comparative group and the experimental groups 1 to 3 showed an obvious decrease. In addition, the decrease of the percentage of cell migration in each of the experimental groups 1 to 3 was significantly greater than that of the percentage of cell migration in the comparative group. In particular, the decrease of the percentage of cell migration in each of the experimental groups 2 and 3 was greater than the decrease of the percentage of cell migration in experimental group 1 and comparative group. These results reveal that the ophthalmic drug delivery carrier according to the present disclosure has an excellent efficacy in inhibiting the migration ability of the HUVECs through grafting with the TAT peptide and the metformin on a surface thereof. Accordingly, the applicant believed that the ophthalmic drug delivery carrier according to the present disclosure may have an antiangiogenic ability, especially the R-PCL-NP having a grafting ratio of the TAT peptide to the metformin of 40:60 or 15:85.

### Example 4. Determination of Permeability of the Ophthalmic Drug Delivery Carrier According to the Present Disclosure

In this example, the applicant utilized an in vivo permeability study to evaluate the permeability of the ophthalmic drug delivery carrier according to the present disclosure. In addition, for comparison purpose, the applicant used different types of cell-penetrating peptides (CPPs) to prepare various ophthalmic drug delivery carriers.

### Experimental Materials

### 1. Preparation of Ophthalmic Drug Delivery Carriers Grafted With Different Kinds of CPPs

Four types of ophthalmic drug delivery carriers (hereinafter abbreviated as “P/M-R-PCL-NP”, “A/M-R-PCL-NP”, “L/M-R-PCL-NP”, and “S/M-R-PCL-NP” respectively), each of which had a certain type of CPP grafted on a surface thereof, were generally prepared with reference to the procedures described in Example 1, except that when carrying out the procedures in Section B of Example 1, four types of CPPs shown in Table 3 below were used to replace the TAT peptide, and when carrying out the procedures in Section D of Example 1, the grafting solution 2 shown in Table 2 was used to prepare a corresponding one of second mixtures.

TABLE 3

| Ophthalmic drug delivery carrier | CPP |
|---|---|
| P/M-R-PCL-NP | Protamine |
| A/M-R-PCL-NP | Poly(arginine) |
| L/M-R-PCL-NP | Poly(lysine) |
| S/M-R-PCL-NP | Poly(serine) |

### Experimental Procedures

First, the T/M-R-PCL-NP 2 obtained in Example 1, and the P/M-R-PCL-NP, the A/M-R-PCL-NP, the L/M-R-PCL-NP, and the S/M-R-PCL-NP obtained in Section 1 of Experimental materials of this example, in appropriate amounts thereof, were respectively dissolved in an acetone solution, thereby obtaining 5test solutions each containing a certain type of ophthalmic drug delivery carrier having a concentration of 10 μg/μL therein.

Afterward, the SD rats were randomly divided into six groups (n=6 rats in each group), including a normal control group, an experimental group, and four comparative groups (i.e., comparative groups 1 to 4). Next, a respective one of the SD rats in each group was subjected to anesthesia using Zoletil® (2.5 mg/kg body weight) and xylazine hydrochloride (1 mg/kg body weight) via intramuscular injection. Subsequently, the 5 test solutions (added with 10 μL of Nile red) each containing the certain type of ophthalmic drug delivery carrier were respectively administered to an eye of the respective one of the SD rats in the experimental group and the four comparative groups via intravitreal injection, as shown in Table 4 below. In addition, the SD rats in the normal control group received no treatment.

TABLE 4

| Group | Ophthalmic drug delivery carrier |
|---|---|
| Normal control group | — |
| Experimental group | T/M-R-PCL-NP |
| Comparative group 1 | P/M-R-PCL-NP |
| Comparative group 2 | A/M-R-PCL-NP |
| Comparative group 3 | L/M-R-PCL-NP |
| Comparative group 4 | S/M-R-PCL-NP |

On the 56^{th} day after start of administration, the SD rats in each group were sacrificed using CO₂, and then retinal tissues were removed from the eyes of the SD rats using a scalpel blade. Afterward, approximately 12 mg of the thus obtained retinal tissues in each group was taken to serve as a tissue sample. Next, the tissue sample was subjected to a fixation treatment with 4% paraformaldehyde (prepared in phosphate buffered saline (PBS)) at room temperature for 2 hours, and then was embedded with paraffin, followed by slicing, so as to obtain a tissue section having a thickness of 5 μm.

Subsequently, the tissue section was stained with 4′,6-diamidino-2-phenylindole (DAPI) solution, and then was observed and photographed using a fluorescence microscope (Axiovert 200M, Carl Zeiss) at a magnification of 10×. The appearance of a red fluorescent light indicated that the certain type of ophthalmic drug delivery carrier had successfully permeated into the retinal tissues. Finally, the mean fluorescence intensity of the tissue section was measured using ImageJ software. The relative fluorescence intensity of each group was calculated using the following Equation (4):

$$H = I / J \qquad ( 3 )$$

where H=relative fluorescence intensity of each group
I=mean fluorescence intensity of each group
J=mean fluorescence intensity of the experimental group

The data thus obtained were analyzed according to the procedures as described in Section 1 of General Experimental Procedures.

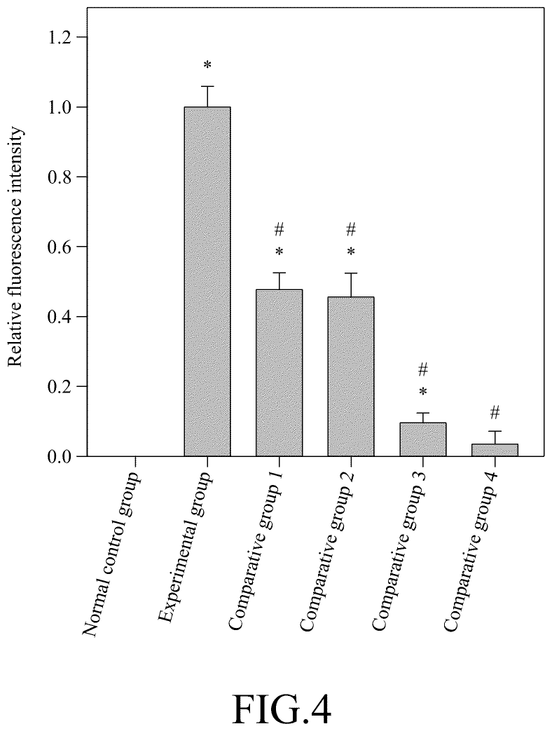

Referring to FIG. 4, it can be seen that compared with the normal control group, the relative fluorescence intensity of each of the experimental group and comparative groups 1 to 3 increased significantly. In addition, the relative fluorescence intensity of the experimental group was significantly higher than that of each of the comparative groups 1 to 3, while there was no significant difference between the relative fluorescence intensity of the comparative group 4 and the relative fluorescence intensity of the normal control group. These results show that compared with the ophthalmic drug delivery carriers prepared by grafting respectively with the 4 kinds of CPPs as mentioned above, the ophthalmic drug delivery carrier according to the present disclosure, which utilizes the TAT peptide to serve as a CPP, exhibits the best retinal permeability, and hence the ophthalmic drugs (i.e., resveratrol and metformin) can effectively penetrate into the retinal tissues, particularly into retinal pigment epithelium (RPE) cells.

### Example 5. Evaluation of the Efficacy of the Ophthalmic Drug Delivery Carrier According to the Present Disclosure in Treating age-Related Macular Degeneration (AMD)

### Experimental Materials

### 1. Preparation of a Resveratrol-Encapsulating PCL Nanoparticle Grafted With a TAT Peptide:

A resveratrol-encapsulating PCL nanoparticle grafted with a TAT peptide (hereinafter abbreviated as “T-R-PCL-NP”) was generally prepared with reference to the procedures described in Example 1, except that when carrying out the procedures in Section D of Example 1, a grafting solution that merely contained the TAT-PEG-COOH (1.5 g) was used.

### 2. Preparation of a Resveratrol-Encapsulating PCL Nanoparticle Grafted With Metformin:

A resveratrol-encapsulating PCL nanoparticle grafted with metformin (hereinafter abbreviated as “M-R-PCL-NP”) was generally prepared with reference to the procedures described in Example 1, except that when carrying out the procedures in Section D of Example 1, a grafting solution that merely contained the Met-PQ-COOH (0.75 g) was used.

3. Preparation of an Avastin®-Encapsulating PCL Nanoparticle Grafted with a TAT Peptide:

An Avastin®-encapsulating PCL nanoparticle grafted with a TAT peptide (hereinafter abbreviated as “T-A-PCL-NP”) was generally prepared with reference to the procedures described in Example 1, except that when carrying out the procedures in Section A of Example 1, the Avastin® was used to replace the resveratrol, and when carrying out the procedures in Section D of Example 1, a grafting solution that merely contained the TAT-PEG-COOH (1.5 g) was used.

### Experimental Procedures

### A. Induction of AMD

Firstly, the SD rats were randomly divided into six groups (n=6 rats in each group), including a normal control group, a pathological control group, an experimental group, and three comparative groups (i.e., comparative groups 1 to 3). Next, the SD rats in each of the pathological control group, the experimental group and the comparative groups 1 to 3 were exposed to a while fluorescent lamp (FHD100ECW, Panasonic) having an illumination of 2000 Ix for 8 hours, once a day for a total period of 30 days, thereby inducing AMD. In addition, the SD rats in the normal control group received no treatment.

### B. Administration of PCL Nanoparticles

First, the T/M-R-PCL-NP 2 obtained in Example 1 and the T-R-PCL-NP, the M-R-PCL-NP, and the T-A-PCL-NP respectively obtained in Sections 1, 2 and 3 of Experimental Materials in this example, in appropriate amounts thereof, were dissolved in an acetone solution, respectively, thereby obtaining 4 test solutions each having a certain type of PCL nanoparticles with a concentration of 10 μg/μL therein.

At the 16^{th} hour after completion of the induction of AMD as described in section A of this example, the 4 test solutions (10 μL per test solution) with 4 different types of PCL nanoparticles containing therein were administered, according to Table 5, into an eye of a respective one of the SD rats in the experimental group and comparative groups 1 to 3, respectively, via intravitreal injection. In addition, 10 μL of PBS was administered into an eye of a respective one of the SD rats in the pathological control group via intravitreal injection, and the SD rats in the normal control group received no treatment.

TABLE 5

| Group | PCL nanoparticles |
|---|---|
| Normal control group | — |
| Pathological control group | PBS |
| Experimental group | T/M-R-PCL-NP 2 |
| Comparative group 1 | T-R-PCL-NP |
| Comparative group 2 | M-R-PCL-NP |
| Comparative group 3 | T-A-PCL-NP |

### C. Assessment of Retinal Function

On the 56^{th} day after start of the administration, a respective one of the SD rats in the normal control group, the pathological control group, the experimental group and the comparative groups 1 to 2 was subjected to anesthesia, followed by placing a subcutaneous needle electrode connected to an amplifier on a neck of each of the SD rats in each group, and then a photic stimulator having a maximum luminance of 90 cds/m² was used to generate electroretinogram (ERG) signals, so as to obtain an ERG pattern of the mice in each group. Afterward, the retinal function of the SD rats in each group was assessed by analyzing the amplitudes (μV) of the a-wave and the b-wave in the ERG pattern of the mice in each group.

The data thus obtained were analyzed according to the procedures as described in Section 1 of General Experimental Procedures.

### D. Determination of Vascular Front Density

After completion of the assessment as described in Section C of this example, the SD rats in each group were sacrificed using CO₂, and then retinal tissues were removed from the eyes of the SD rats using a scalpel blade. Next, the retinal tissue of each rat was placed on slides, and was then subjected to fixation treatment using 4% methanol at room temperature for 2 hours, followed by staining using Alexa Fluor® 568-conjugated isolectin GS-IB4 (Invitrogen) (1:1000). Finally, the stained retinal tissue was observed and photographed using the fluorescence microscope (Axiovert 200M, Carl Zeiss) at a magnification of 10×. The vascular front density (%) of the retinal tissue of each rat was determined using ImageJ software.

The data thus obtained were analyzed according to the procedures as described in Section 1 of General Experimental Procedures.

### Results

### A. Assessment of Retinal Function

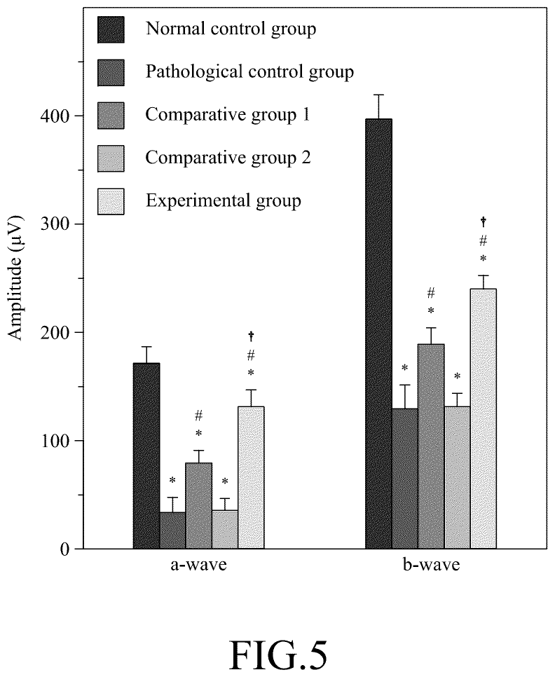

Referring to FIG. 5, it can be seen that the amplitudes determined in both the a-wave and the b-wave of the pathological control group were significantly decreased compared with those of the normal control group, indicating that AMD was successfully induced and retinal function was truly impaired. Moreover, compared with the pathological control group, the amplitudes determined in both the experimental group and the comparative group 1 were significantly increased, and the amplitudes in the experimental group were much higher than those of the comparative groups 1. However, the amplitudes determined in the comparative group 2 showed no statistical difference compared with those determined in the pathological control group. These results show that the T/M-R-PCL-NP (i.e., the ophthalmic drug delivery carrier according to the disclosure) can effectively alleviate AMD and improve the retinal function, and the efficacy thereof is significantly better than those of the T-R-PCL-NP and the M-R-PCL-NP.

### B. Determination of Vascular Front Density

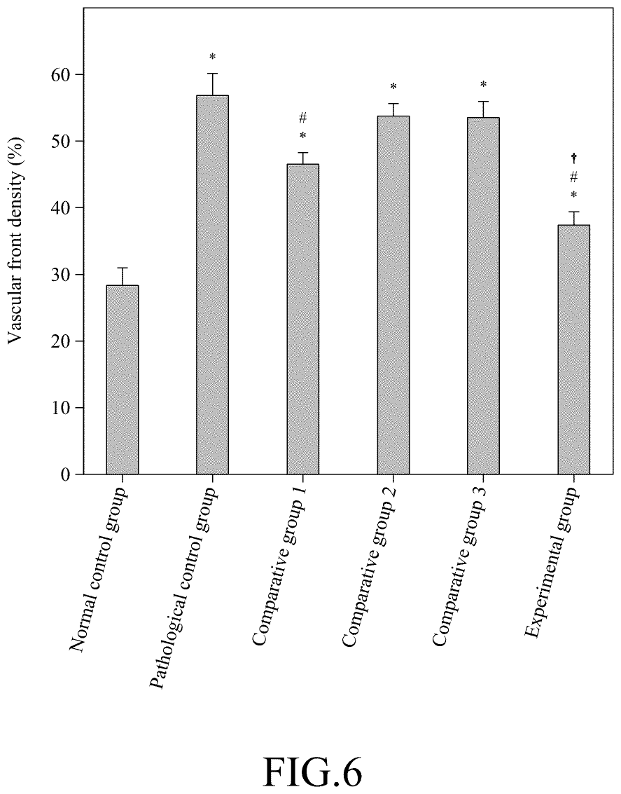

Referring to FIG. 6, it can be seen that compared with the normal control group, the vascular front density of the pathological control group was significantly increased, indicating that AMD was successfully induced to allow abnormal proliferation of blood vessels. In addition, compared with the pathological control group, the vascular front densities measured in both the experimental group and the comparative group 1 were significantly reduced, and the vascular front density measured in the experimental group was much lower than the vascular front density measured in the comparative group 1. However, there was no statistical difference between the vascular front density measured in each of the experimental groups 2 and 3 and the vascular front density measured in the pathological control group. These results show that the T/M-R-PCL-NP (i.e., the ophthalmic drug delivery carrier according to the disclosure) can effectively alleviate AMD and inhibit abnormal vascular proliferation, and the efficacy thereof is significantly better than those of the T-R-PCL-NP, the M-R-PCL-NP, and the T-A-PCL-NP.

In sum, the ophthalmic drug delivery carrier according to the disclosure, by grafting the TAT peptide on the surface thereof, exhibits superb retinal permeability, so that two immiscible drugs, i.e., the resveratrol encapsulated therein and the metformin grafted on the surface thereof, can be delivered into the retinal tissue simultaneously and effectively, thereby synergistically exerting the effect of alleviating AMD.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An ophthalmic drug delivery carrier, comprising:
a resveratrol-encapsulating poly(ε-caprolactone) (PCL) nanoparticle;
metformin grafted on a surface of the resveratrol-encapsulating PCL nanoparticle; and
a transacting activator of transcription (TAT) peptide grafted on the surface of the resveratrol-encapsulating PCL nanoparticle.

2. The ophthalmic drug delivery carrier as claimed in claim 1, wherein the resveratrol-encapsulating PCL nanoparticle has a particle size ranging from 80 nm to 150 nm.

3. The ophthalmic drug delivery carrier as claimed in claim 1, wherein a grafting ratio of the TAT peptide to the metformin ranges from 10:90 to 90:10.

4. A method for preparing an ophthalmic drug delivery carrier as claimed in claim 1, comprising:
mixing an emulsion containing resveratrol with PCL and a surfactant for double emulsification so as to form a mixture, followed by subjecting the mixture to centrifugation so as to obtain a precipitate;
subjecting the precipitate to an amination reaction with a diamine solution, so as to obtain a resveratrol-encapsulating PCL nanoparticle;
subjecting a TAT peptide to a carboxylation reaction so as to obtain a TAT peptide having a carboxyl group;
subjecting metformin to a carboxylation reaction so as to obtain metformin having a carboxyl group; and
subjecting the resveratrol-encapsulating PCL nanoparticle to a grafting reaction with the TAT peptide having the carboxyl group and the metformin having the carboxyl group, thereby obtaining the ophthalmic drug delivery carrier,
wherein in the grafting reaction, the resveratrol-encapsulating PCL nanoparticle, the TAT peptide having the carboxyl group, and the metformin having the carboxyl group are present in a weight ratio ranging from 1:1.5:0.15 to 1:1.5:1.5.

5. The method as claimed in claim 4, wherein in the grafting reaction, the resveratrol-encapsulating PCL nanoparticle, the TAT peptide having the carboxyl group, and the metformin having the carboxyl group are present in a weight ratio of 1:1.5:0.75.

6. A method for alleviating macular degeneration, comprising administrating to a subject in need thereof a pharmaceutical composition containing an ophthalmic drug delivery carrier as claimed in claim 1.

7. The method as claimed in claim 6, wherein the pharmaceutical composition is in a dosage form for intraocular administration or topical ophthalmic administration.


SEQUENCE LISTING (2 sequences)

SEQ ID NO: 1 - protein, 9 aa, synthetic construct
  source 1..9: note = cell penetrating peptide
RKKRRQRRR

SEQ ID NO: 2 - protein, 12 aa, synthetic construct
  source 1..12: note = MMP-sensitive peptide
GGGPQGIWGQGK